(19) United States
(12) Patent Application Publication
(10) Pub. No.: US 2025/0232250 A1
(43) Pub. Date: Jul. 17, 2025

(54) APPARATUS FOR GENERATING A CHEMICAL PRODUCT PASSPORT

(71) Applicant: BASF SE, Ludwigshafen am Rhein (DE)

(72) Inventor: Henning SCHWABE, Ludwigshafen (DE)

(21) Appl. No.: 18/723,309

(22) PCT Filed: Dec. 19, 2022
(86) PCT No.: PCT/EP2022/086787

§ 371 Date: Jun. 21, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 10/0833; G06Q 50/04; G16C 20/80; G16C 20/90

(52) U.S. Cl. CPC G06Q 10/0833; G06Q 50/04; G16C 20/80; G16C 20/90

(57) ABSTRACT

The invention refers to an apparatus for generating a chemical product passport for a chemical product. One or more computing processors of the apparatus 130 are configured to perform the following steps. Product data referring to information related to the product and/or one or more pre-products utilized in the production process of the product is received. Further, a semantic model comprising data related to at least a part of the product data is accessed. The chemical product passport 135 is then generated based on the product data and the semantic model, wherein the chemical product passport comprises a decentralized identifier identifying the product, at least a part of the product data and a relation between at least a part of the product data provided by the chemical product passport and the semantic model.

### FIELD OF THE INVENTION

The invention relates to an apparatus, a method and a computer program product for generating a chemical product passport. Further, the invention relates to a chemical product passport generated by the apparatus, method and computer program product and to a semantic model utilizable for generating a chemical product passport.

### BACKGROUND OF THE INVENTION

Today, products are not only produced in a single production process by a single production entity, but often the production of a product is spread to different production entities that can be part of the same industrial compound or can be located at completely different locations. This is in particular true in cases of recycling processes in which a product is produced such that at least parts of the products can again be utilized as pre-products in a production of another product. Due to these complex production processes and often long chains of production steps associated with the product, it is often difficult to not only clearly identify the product itself but also the respective characteristics of the product in its present state. A known identity and characteristic of a product, however, has a huge impact on respective production processes utilizing this product. Thus, it would be advantageous to improve the knowledge available for a product in a secure and computational resource less intensive way, in particular, for recycling production processes.

### SUMMARY OF THE INVENTION

It is an object of the present invention to provide an apparatus that allows to improve the knowledge on a specific product in a secure and computational less expensive manner.

In a first aspect of the present invention an apparatus for generating a chemical product passport for a chemical product, wherein the chemical product passport is indicative of a digital representation of data associated with the chemical product, wherein the apparatus comprises one or more computing processors configured to perform the followings steps a) receiving product data referring to information related to the product and/or one or more pre-products utilized in the production process of the product, b) accessing a semantic model comprising data related to at least a part of the product data, c) generating the chemical product passport based on the product data and the semantic model, wherein the chemical product passport comprises a decentralized identifier identifying the product, at least a part of the product data and a relation between at least a part of the product data provided by the chemical product passport and the semantic model, and d) providing the generated product passport.

Since the generated chemical product passport comprises not only a decentralized identifier identifying the product and part of the product data, but also a relation between at least a part of the product data and a semantic model that comprises data related to at least a part of the product data, a chemical product cannot only be clearly identified and respective information about the chemical product can be provided in a secure manner, but also if initial data related to the product data is needed, for instance, additional specifications, utilized test procedures, certificates, etc., these do not have to be all stored together with the product passport but can be easily accessed in a fast and easy manner via the product passport. Thus, a such generated product passport allows for a secure and computational inexpensive manner of providing information related to a product.

Generally, the apparatus can be realized in form of any general or dedicated software and/or hardware comprising one or more processors that are configured accordingly. In particular, the apparatus can also be realized in form of a distributed computing in which the one or more processors are distributed over different computers or computing systems, for instance, in a cloud computing or network computing environment. The apparatus is configured for generating a chemical product passport. A chemical product passport is indicative of a digital representation of data associated with a chemical product. The digital representation can refer to any representation of data that allows for accessing the respective data or parts thereof.

Generally, the chemical product for which the passport is generated refers to any chemical product obtained from a chemical reaction either performed naturally leading to a natural chemical product, or industrially leading to an industrial chemical product. Natural chemical products encompass any chemical substance that is naturally occurring, i.e. any unprocessed chemical substance that is found in nature, such as chemicals from plants, micro-organisms, animals, the earth and the sea or any chemical substance that is found in nature and extracted using a process that does not change its chemical composition. Natural chemical products may include biologicals like enzymes as well naturally occurring inorganic or organic chemical products. Natural chemical products can be isolated and purified prior to their use or they can be used in unisolated and/or unpurified form. Industrial chemical products obtained from industrial chemical reactions may be any inorganic or organic chemical product obtained by reacting inorganic and/or organic chemical reactants in industrial processes. The inorganic and organic chemical reactants may be naturally occurring chemical products or can be chemical products obtained from chemical reactions.

Chemical reactions may include any chemical reaction commonly known in the state of the art in which the reactants are converted to one or more different chemical products. Chemical reactions may involve the use of catalysts, enzymes, bacteria, etc. to achieve the chemical reaction between the reactants.

The one or more computing processors are configured to receive product data referring to information related to the product and/or one or more pre-products utilized in the production of the process of the product. Generally, product data refers to data that is associated with the chemical product. In particular, chemical product data can comprise any data related to a property of the chemical product and/or data related to the use of the chemical product. Such property may be a static or a dynamic property. A static property may be a property constant over time e.g. melting point, boiling point, density, hardness, flammability or the like. A dynamic property may be a property that changes over time e.g. shelf life, pH value, color, reactivity. A property of the chemical product may include performance properties, chemical properties, such as flammability, toxicity, acidity, reactivity, heat of combustion and/or physical properties such as density, color, hardness, melting and boiling points, electrical conductivity or the like. Data related to the use of the chemical product may include data related to further processing of the chemical product, for example by using the chemical product as reactant in further one or more chemical reactions and/or data related to the use of the chemical product, for example data related to the use of the chemical product in a treatment process and/or within a manufacturing process. Chemical product data may include chemicals data, emission data, recyclate content, bio-based content and/or production data. Thus, the chemical product data can generally be regarded as data that can influence the further processing of a chemical product and/or its utilization in other products.

In a further step, the one or more computing processors are configured to access a semantic model comprising data related to at least a part of the product data. Generally, the data related to at least a part of the product data can also be data that is related to the product. However, the data can also only be related to the product via the product data. An example for this could be general data related to a testing procedure of an inflammation temperature of a chemical product. The inflammation temperature itself refers to data that is directly related to the product, in this case the value of this data is even a characteristics of the product. The general testing procedure with which the inflammation temperature has been determined, however, is not directly related to the product, but is related to the combustion point that has been determined using this respective general testing procedure. Accordingly, for cases in which the combustion point has been determined using the respective testing procedure for a chemical product, the respective testing procedure is data related to the product data of the chemical product and also indirectly related to the chemical product via the product data. Thus, in particular, the semantic model comprises data that is more general than the specific product data that is in most cases specific to the chemical product and thus can connect a specific product data to more general information. However, the semantic model can also comprise other data that are not related to the product data, for instance, in the example above, can also comprise information on other combustion point test procedures that have not been used for determining the combustion point of the chemical product. Thus, generally, the semantic model can be an extensive database storing a plurality of different information from which at least a part is related to at least a part of the product data.

A semantic model is generally a data model that is based on a semantic based database description and structuring formalism. In particular, the semantic model is a conceptual data model, i.e. a data model template, including semantic information that stores and structures data also based on the meaning of the data such that the relation to the real world becomes accessible. For example, semantic data models are often used as “meta models” or “templates of data models” to describe databases but in some cases also in a modelling language library for e.g. PYTHON that is converted into executable code. Generally, a data model provides a framework for organizing and representing data. The data model can be viewed as a set of rules and/or constraints for the arrangement of the data that defines permissible data structures that are built within the physical storage device. A data structure can be seen as a physical implementation of a data model's logical organization, and the data structure occurs as physical interrelationships among stored bits of data in the memory. The data structure can determine how the data processing system can interact with the datastore, including how pieces of data can be deposited, queried, addressed, and/or retrieved.

Preferably, the semantic model comprises a graph database in which the data related to at least part of the production data is organized. A graph database generally comprises a database that uses graph structures with nodes, edges and properties to represent and store data, wherein data items, here predefined product data items, are stored related to the collection of nodes and edges of the graph database. In this context, edges of the graph database represent a relationship between nodes connected by the respective edges. Nodes represent items of interest for which respective relations are tracked by the graph database. Moreover, properties refer to information related to a respective note item. Thus, a graph database allows not only to store data items but also to store relationships between the data items and properties of the data items such that the data is linked together in a context sensitive way such that respective complex data structures can be visualized and queried.

Further, the chemical product passport is generated based on the product data and the semantic model. Generally, a chemical product passport comprises a digital representation of at least a part of the chemical product data. The digital representation can include a representation for accessing the chemical product data or part thereof, for instance, an ID of the chemical product data under which this data can be found in a database, or a link to the respective data. The digital representation can also include a representation of chemical product data or parts thereof, for instance, a list or table of the information provided by the data including the data itself.

In particular, the chemical product passport comprises decentralized identifier identifying the product. Further, at least a part of the product data and a relation between at least a part of the product data provided by the chemical product passport and the semantic model. A decentral identifier can be any unique identifier uniquely associated with the data owner and chemical product data. The decentral identifier may include a Universally Unique IDentifier (UUID) or a Digital IDentifier (DID). The decentral identifier can be issued by a central or decentral identity issuer. The decentral identifier can include authentication information. Via the decentral identifier and its unique association with the data owner and chemical product data access to the chemical product data can be controlled by the data owner. This contrasts with central authority schemes, where identifiers are provided by such central authority and access to data is controlled by such central authority. Decentral in this context refers to the usage of the identifier in implementation as controlled by the data owner.

The relation between at least a part of the product data provided by the chemical product passport and the semantic model can be any kind of data relation that allows for an association between at least a part of the product data and the semantic model. Preferably, the relation allows for an accessing of the semantic model via the related product data. In particular, product data for which related data is present in the semantic model is linked together. For example, the link can allow to access the related data to the respective product data upon an interaction with the respective product data. Storing only the relation between at least a part of the product data and the semantic model allows to enrich the chemical product passport with the variety of information provided by the semantic model related to the product data without the need of storing this data in the chemical product passport itself.

Further, the generated product passport can then be provided, for instance, together with the product from a respective data owner, for instance, a producer of the product, or from a respective data consuming or data providing service. A data owner can be any entity that generates data. The generating node may be coupled to the entity owning physical products from or for which data is generated. The data may be generated by a third-party entity on behalf of the entity owning physical products from or for which data is generated. Generally, a data consuming service can refer to computer-executable instructions for accessing and/or processing data, such as chemical product data, associated with the data owner. A data providing service can refer to computer-executable instructions for providing and/or processing data, such as chemical product data, associated with the data owner for accessing and/or processing by a data consuming service.

Generally, the providing can refer to storing the generated product passport on a respective accessible storage. However, the providing can also refer to providing the generated product passport for further processing. In particular, it is preferred that the apparatus further comprises a product passport interface, wherein the product passport interface is configured to provide an interface to the data stored in the chemical product passport, in particular, the decentralized identifier, at least a part of the product data, and the relation. In particular, the product passport interface is configured to allow to access via the relation provided by the chemical product passport the semantic model and the related data in the semantic model. Preferably, the controlling signals for controlling a production of a product using the chemical product are generated based on the product passport, in particular, based on the information provided by the product passport. For example, the control signals can cause a production plant to utilize respective production parameters that take the properties of the chemical product as stored on the production passport into account. For instance, based on production properties, or even on a date of production of the chemical product as stored in the product passport the amount of the utilized chemical product can be amended.

In an embodiment, the generating of the chemical product passport comprises selecting the product data provided by the chemical product passport based on a received request, wherein the received request is translated into a graph pattern request and the graph database of the semantic model is filtered for the graph pattern corresponding to the graph pattern request and the chemical data provided by the chemical product passport is selected based on the filtered graph pattern. In this case, the chemical product passport does not comprise fixed product data but is generated based on a request with the chemical product data being selected based on the request. In particular, since in this embodiment the semantic model is a graph database, respective requests that can be provided, for instance, by a user, are translated into a graph pattern request and the result of the request refers to a filtered graph pattern, i.e. the graph pattern resulting from the graph pattern request. The chemical data provided by the chemical product passport is then selected based on the filtered graph pattern, for instance, information stored in the filtered graph pattern is utilized as selected chemical data provided by the chemical product passport.

In an embodiment, the chemical product comprises a physical identifier and the generating of this chemical product passport comprises assigning the physical identifier to the decentral identifier, wherein the product data refers to data collected in association with the physical identifier of the chemical product. The physical identifier can be any kind of physical identifier, for instance, a barcode, an RFID chip, a branding, a watermark, a stamp, a seal, etc. Moreover, the physical identifier can also be a specific characteristic or a combination of specific characteristics of the product that allows to uniquely identify the physical product. Thus, assigning the physical identifier that uniquely allows to identify the chemical product with the decentral identifier allows for a secure and unique identification of the chemical product via the decentral identifier. In particular, it can be ensured that the chemical product passport is indeed the chemical product passport of the respective chemical product. Tempering and unauthorized amendments with respect to the identification of the product can in this way be prevented or will at least be noted, for instance, when checking the physical identifier that is associated with the decentralized identifier. Preferably, the assigning thus refers to a storing of the assignment, for instance, also as part of the chemical product passport. However, the assignment can also be stored in any other way, preferably in form of a block chain that allows for a temper-resistant or tamper-proof and secure association that is accessible by a plurality of parties. Thus, also, for instance, if the product changes its ownership a few times, the association between the physical identifier and the decentralized identifier of the chemical product passport is secured and cannot be changed unauthorized by one of the parties.

In an embodiment, the decentral identifier is further associated with a data schema and the chemical product passport is generated to comprise at least a part of the data schema and/or a data schema ID. Generally, a semantic model can be provided in a plurality of different types of data schemas, thus, accessing a respective semantic model is computationally easier if a respective data schema or a data schema ID is associated with the respective chemical product passport. In particular, the knowledge of the utilized data schema allows to formulate a request for accessing the semantic model accordingly.

In an embodiment, the chemical product passport is associated with one or more authorization mechanisms that refer to the decentral identifier, the chemical product data provided by the chemical product passport and/or the access to the semantic model. In particular, the authorization mechanisms can regulate the access and other options that a user has with respect to the chemical product passport. For example, the authorization mechanism can regulate that a specific person, company or other party has access only to specific chemical product data and, for instance, has no access to the semantic model, i.e. cannot access the semantic model via the relations stored in the chemical product passport. In contrast thereto, other parties can be provided by the authorization mechanism with the authorization to have full access to all features provided by the chemical product passport. Even more important is the authorization mechanism when it comes to the possibility to amend or expand the chemical product passport, for instance, a part of the chemical product data stored with respect to the chemical product passport or adding product data that is stored on the chemical product passport optionally together with respective relations to the semantic model.

In an embodiment, the at least a part of the product data includes at least one of chemical product declaration data, chemical product safety data, material information and a certificate of analysis data associated with at least one physical entity of the chemical product. The material information can, for instance, refer to at least one of physical properties, material performance, technical applications, and other data related to one or more materials of the chemical product.

In a further aspect of the invention, a chemical product passport indicative of a digital representation of data associated with the chemical product is presented, wherein the chemical product passport comprises a decentralized identifier identifying the product, at least a part of product data and a relation between at least a part of the product data provided by the chemical product passport and a semantic model, wherein the product data refers to information related to the product and/or one or more pre-products utilized in the production process of the product, and wherein the semantic model comprising data related to at least a part of the product data. In particular, the chemical product passport is a chemical product passport generated by the above described apparatus.

In a further aspect, a semantic model is presented utilizable for generating a chemical product passport for a chemical product according to any of claims 1 to 9, wherein the semantic model comprising data related to at least a part of product data referring to information related to the product and/or one or more pre-products utilized in the production process of the product, wherein the semantic model is configured such that a relation in the chemical product passport allows access to the semantic model.

In a further aspect, a computer-implemented method is presented for generating a chemical product passport, wherein the chemical product passport is indicative of a digital representation of data associated with a chemical product, wherein the method comprises a) receiving product data referring to information related to the product and/or one or more pre-products utilized in the production process of the product, b) accessing a semantic model comprising data related to at least a part of the product data, c) generating the chemical product passport based on the product data and the semantic model, wherein the chemical product passport comprises a decentralized identifier identifying the product, at least a part of the product data and a relation between at least a part of the product data provided by the chemical product passport and the semantic model, and d) providing the generated product passport.

In a further aspect, computer program product is presented for generating a chemical product passport comprising program code means for cause an apparatus as described above to perform the method as described above. In particular, the computer program product comprises program code means that causes the apparatus as described above to perform the method as described above to generate the chemical product passport as described above.

It shall be understood that the apparatus as described above, the method as described above and the computer program product as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 shows schematically and exemplarily an embodiment of a system comprising an apparatus for generating a chemical product passport,

FIG. 2 shows schematically and exemplarily a flowchart of an embodiment of a method for generating a chemical product passport,

FIG. 3 shows schematically and exemplarily an example for a realization of a data environment for the providing of the product passport, and

FIG. 4 shows a schematic illustration of providing access to a product passport of a data providing service in the International Data Space (IDS) architecture to a data consuming service.

### DETAILED DESCRIPTION OF EMBODIMENTS

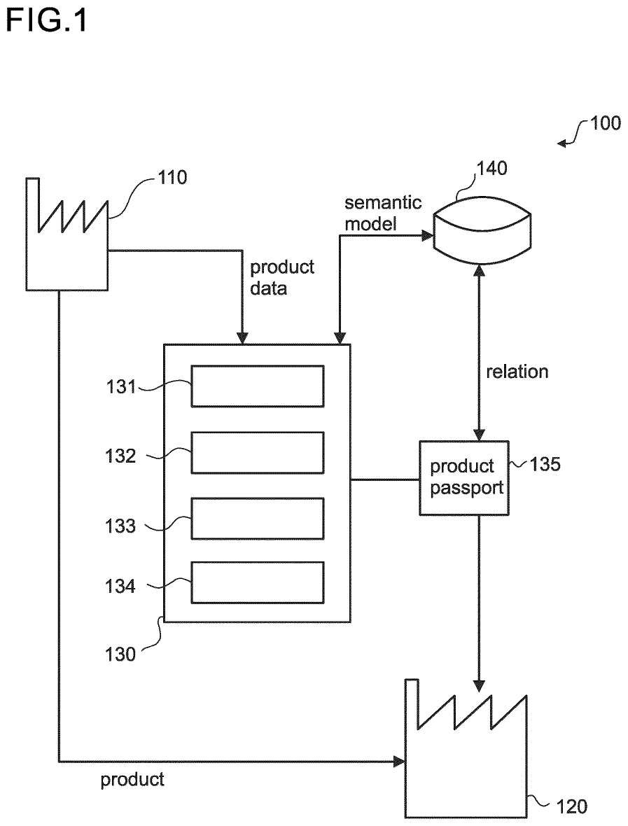

FIG. 1 shows schematically and exemplarily a system 100 comprising an apparatus 130 for generating a product passport. Further, the system can encompass an interface 110 with an industrial production plant producing the chemical product, a storage unit 140 for storing a semantic model and an interface 120 for interfacing with a customer industrial production plant.

In this exemplary embodiment, an industrial production plant generates a chemical product. Generally, the production plant can refer to any technical infrastructure that is used for an industrial purpose. The industrial purpose may be manufacturing or processing of one or more industrial products, i.e., a manufacturing process or a processing performed by the industrial plant. In this example, the industrial purpose refers to the production of a specific product. The specific product can, for example, be any physical product such as a chemical, a biological, a pharmaceutical, a food, a beverage, a textile, a metal, a plastic, a semiconductor. Accordingly, the production plant 130 may be one or more of a chemical plant, a process plant, a pharmaceutical plant, a fossil fuel processing facility such as an oil and/or a natural gas well, a refinery, a petrochemical plant, a cracking plant, and the like. The production plant can even be any of a distillery, an incinerator, or a power plant. The production plant can even be a combination of any of the examples given above. For performing the production process the production plant comprises a technical infrastructure which can be controlled by control parameters implemented by the process control system into the technical infrastructure. The technical infrastructure may comprise equipment or process units such as any one or more of a heat exchanger, a column such as a fractionating column, a furnace, a reaction chamber, a cracking unit, a storage tank, a precipitator, a pipeline, a stack, a filter, a valve, an actuator, a transformer, a circuit breaker, a machinery e.g., a heavy duty rotating equipment such as a turbine, a generator, a pulverizer, a compressor, a fan, a pump, a motor, etc. Moreover, the production plant typically comprises a plurality of sensors that allow to measure operational parameters of the technical infrastructure but also characteristics of the produced chemical product. Moreover, the production plant can also comprise one or more testing facilities configured to perform one or more tests with respect to one or more products or pre-products, for example, to determine one or more properties, verify such properties, or certify properties of the product or a preproduct.

The production plant can be part of a plurality of production plants. For example, the plurality of production plants can refer to a compound of at least two production plants having at least one common industrial purpose. Specifically, a plurality of production plants may comprise at least two, at least five, at least ten or even more production plants being physically and/or chemically coupled. The plurality of production plants may be coupled such that the production plants forming the plurality of production plants may share one or more of their value chains, educts and/or products. The plurality of production plants may also be referred to as a compound, a compound site, a Verbund or a Verbund site. Further, the value chain production of the plurality of production plants via various intermediate products to an end product may be decentralized in various locations, such as in various production plants, or integrated in the Verbund site or a chemical park. Such Verbund sites or chemical parks may be or may comprise one or more production plants, where products manufactured in the at least one production plant can serve as a feedstock for another production plant.

The such produced chemical product is then provided to a consumer, in this case a further industrial plant producing a product utilizing the chemical product. In addition to providing the product physically to a respective consumer, the production plant comprises or utilizes apparatus 130. The apparatus 130 is configured for generating a product passport and is realized as any kind of computing system comprising one or more processors that are configured for providing the following logical units. In particular, the apparatus 130 comprises as logical units a receiving unit 131, an accessing unit 132, a generating unit 133 and a providing unit 134. The receiving unit 131 is configured for receiving product data for the respective chemical product, for instance, directly provided via the interface 110 from the production plant. The product data comprises information that is related to the product and/or one or more pre-products utilized in the production process of the product. For example, the product data can refer to the production parameters utilized for producing the chemical product, characteristics and/or properties measured for the final chemical product, for example, in a testing facility, specifications and applicabilities of the chemical product, security related information like hazard information related to the chemical product, etc.

Further, the apparatus 130 is configured to access, for instance, via the accessing unit 132, a storage unit 140 on which a semantic model is stored. The storage unit 140 is preferably a cloud storage or at least some kind of distributed storage. However, the storage unit 140 can also be any kind of storage unit and can, for instance, be provided by the production plant. The semantic model is preferably a graph database in which data related to at least a part of the product data of the chemical product is organized. In particular, a graph database allows not only to store data but also to store the relation and connections between different data entries based on their meaning, in particular, based on semantic information with respect to the data entries. This allows to easily access information semantically related to a specific data entry. Generally, the utilized semantic model stores a plurality of specific and general information that can be related to any kind of chemical product, wherein in particular also the relation between the respective information is provided by the semantic model. For example, the semantic model can store information on a plurality of testing procedures for properties of chemical products that are related semantically to the respective property, wherein the respective property can further be related to information about possible applications of chemical products with this property, and/or to information on certificates that are based at least partly on this property.

The generation unit 133 is then configured to generate a chemical product passport for the chemical product based on the product data and the semantic model. In particular, the chemical product passport comprises a decentralized identifier identifying the product. The decentralized identifier in particular comprises a unique identifier, for instance, a universal unique identifier (UUID) or a digital identifier (DID), and may be generated or provided by a central or decentral identity provider. For example, in this case the identity provider can be the producer of the chemical product. Preferably, the decentralized identifier is further associated with a physical identifier of the chemical product. The physical identifier can be any identifier that is permanently attached to or part of the chemical product. For example, the physical identifier can be data stored on an RFID chip, a number or other identification sign permanently integrated into the chemical product, a respective combination of properties of the chemical product that is unique, like a chemical fingerprint, for the respective chemical product or a batch of the chemical product, etc. Preferably, the association of the physical identifier with the decentral identifier provided by the chemical product passport is stored, for instance, as part of the chemical product passport itself or independent of the chemical product passport, for instance, in a block chain that additionally prevents a manipulation of this association. Generally, the physical identifier associated with the decentral identifier allows for a secure and unique identification of the chemical product both physically and also with respect to the data related to the chemical product. Manipulations of the chemical product passport or the physical product or the association of both thus become less likely.

Further, the chemical product passport is generated to comprise at least a part of the product data. Which part of the product data is utilized when generating the chemical product passport can depend on a specific application. For example, if the chemical product passport is generated for being utilized by the customer production plant in a further production, the part of the product data provided by the chemical product passport can refer to respective properties of the chemical product determined in one or more testing procedures that can be advantageous to know for the further production processes in which the chemical product can be utilized. Moreover, also hazard information or other security details can be provided as part of the chemical product passport, for instance, to inform public authorities, for example, during logistic procedures. Generally, the product data provided as part of the chemical product passport can be selected by the producer of the product, for instance, by simply providing only the selected product data to the apparatus 130 or can also be selected by a recipient of the product passport, for instance, the customer production plant.

For example, if the product data is also stored as part of the semantic model, a customer can provide a specific request for information relevant for the customer, wherein this request can be translated into a graph pattern or other data model that can be utilized for requesting the semantic model and the product data provided in the generated chemical product passport can then be based on the result of the respective request, in particular, on a filtered graph pattern as result of a graph pattern request in case of a graph database. This allows to customize the information provided by the product passport directly to the respective needs and interests of a consumer and user of the chemical product passport.

Further, the chemical product passport comprises a relation between at least a part of the product data provided by the chemical product passport and the semantic model. Generally, the relation preferably provides access to the semantic model via the relation. For example, the relation can refer to any kind of linking between at least a part of the product data provided by the chemical product passport and at least some part of the semantic model. For example, in a simple embodiment, the relation is provided simply as a link that can be utilized to access via the respective product data in the chemical product passport the related data in the semantic model. However, also more complex data relations can be utilized. For example, the relation can be a kind of request on the semantic model filtering the semantic model to provide only the requested information. Thus, the relation provided by the chemical product passport allows to access the semantic model, in particular, a specific part of the semantic model with information related to a respective product data via the chemical product passport. Accordingly, more information than can be stored on the chemical product passport can be made available to a user of the chemical product passport without having to directly store the information with the product passport which could lead to an increase in computational resources needed for using the chemical product passport, for instance, for copying, accessing and/or storing the chemical product passport. For example, if the chemical product passport comprises a value for a specific property of the chemical product, for instance, a boiling temperature, the relation can link the specific value of the boiling temperature to respective information on boiling temperatures in the semantic model. For example, the link can relate the specific value of the boiling temperature to information on a testing procedure for the boiling temperature stored on the semantic model that is utilized for determining the boiling temperature of the respective chemical product. From this starting information in the semantic model, a user can, for instance, also access related information in the semantic model like other possible procedures or, for instance, information on regulations concerning these testing procedures. Further, the specific value of the boiling temperature can also be related to information in the semantic model that is concerned with possible applications of this boiling temperature in production processes such that respective information also becomes available to a user of the chemical product passport.

The providing unit 134 can then be configured to provide the generated chemical product passport 134 to a respective storage, for instance, to a decentralized storage unit which can be accessed by a respective user of the chemical product passport or directly to a storage provided by the user of the chemical product passport. In this context, the chemical product passport is provided such that the relation to the semantic model is not lost, i.e. that the chemical product passport is still part of the same computational network that allows access to the respective semantic model via the chemical product passport. For providing a chemical product passport with some platform independence, the decentralized identifier of the chemical product passport can be further associated with a data schema that indicates and defines how the semantic model can be accessed.

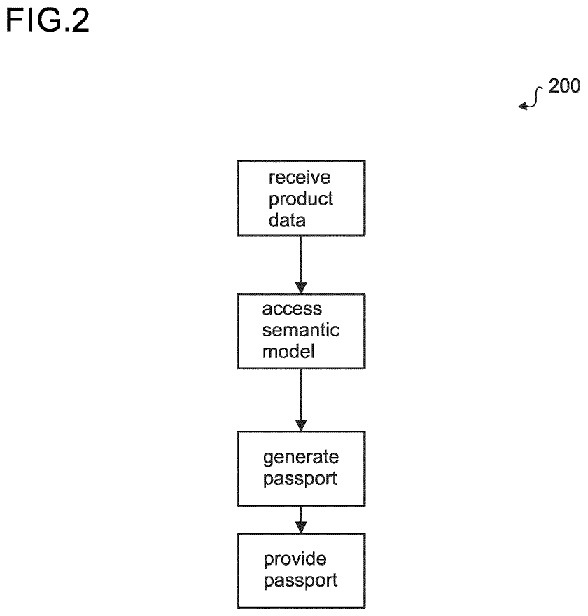

FIG. 2 shows schematically and exemplarily a method 200 for generating a chemical product passport. Generally, the method 200 can be performed, for instance, by an apparatus 130 as described with respect to FIG. 1. Thus, also the more detailed description provided with respect to FIG. 1 can be applied to the method as described in the following. In a first step, the method 200 comprises receiving product data referring to information related to the product and/or one or more pre-products utilized in the production process of the product. Further, the method comprises accessing a semantic model comprising data related to at least a part of the production data. Moreover, in a following step, the method comprises generating the chemical product passport based on the product data and the semantic model. A more detailed description of the generation and the chemical product passport can be found in the examples described above with respect to FIG. 1. In a last step, the generated product passport is provided.

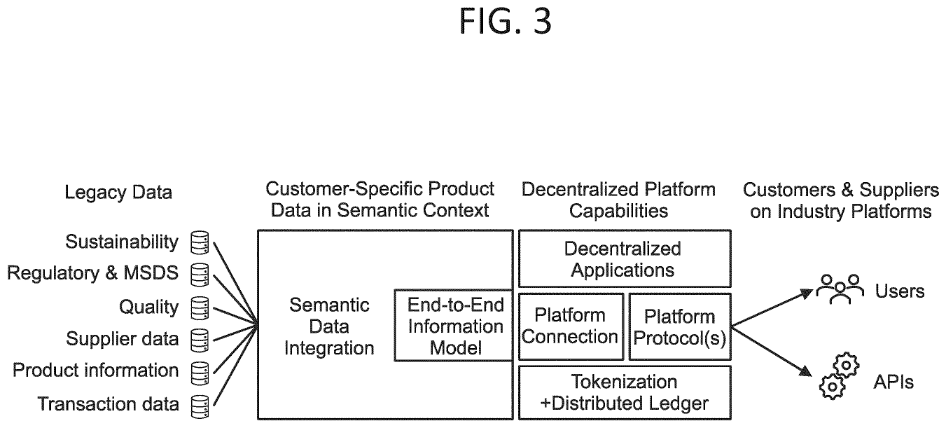

An example for a realization of a data environment for the providing of the product passport is illustrated schematically and exemplarily in FIG. 3. Today business data relevant for communication and supply chain coordination with suppliers and customers is often stored in data silos shown on the left part of FIG. 3. Semantic data models allows to create and maintain context across the such siloed data or other data related to the production process of a product. A respective information model that includes knowledge on the relation of information in the siloed data can be utilized as design guide on a higher level of abstraction above the set of semantic data models. Thus the whole data provision application, for example, the providing of data to a customer, can be designed to maintain context of information as data is being processed into a format mandated by the customer-specific chemical product passport. Preferably, an information model, optionally, also in semantic form, is utilized to cover or encompasses a plurality of semantic data models. Thus, the semantic data model architecture allows for the following advantages. In particular, data context can be maintained across different data silos for the chemical product passport benefitting the customer. Moreover, the context for creating, maintaining, and developing an application serving the chemical product passport can be improved.

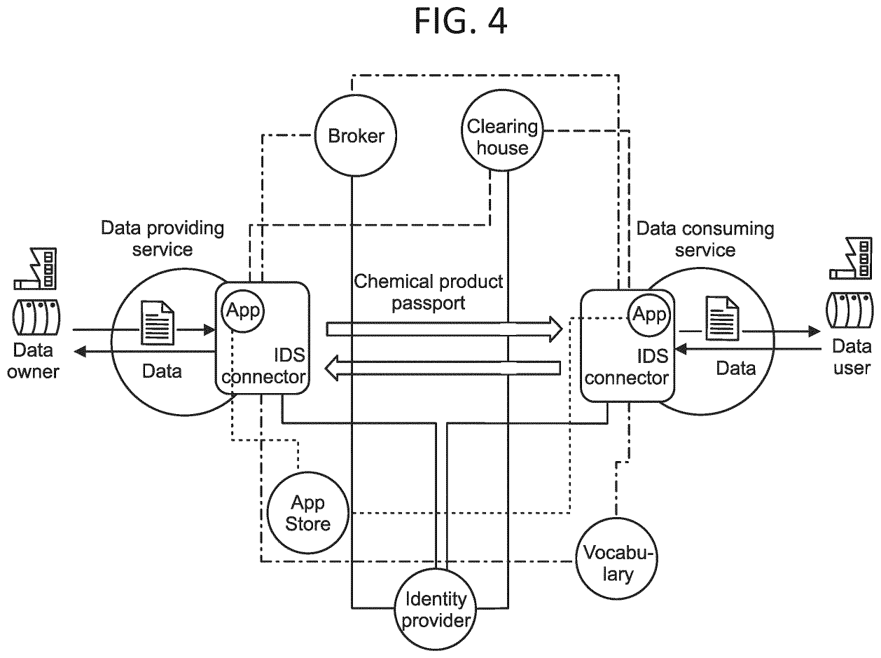

FIG. 4 shows a schematic illustration of providing a chemical product passport from a data providing service via an IDS architecture. The data providing service may provide chemical product data to a data consuming service. The following IDS components may be executable components in the “IDS infrastructure”: Data Connector, App Store, Identity Provider, Vocabulary Provider, Broker, Clearing House. Data may be exchanged between an interface of a data owner and an interface of a data user via a data providing service and a data consuming service. The data providing service and the data consuming service are each connected to the data connector to allow a secure and trusted data exchange. The data provided by the interface of the data owner may be associated with authorization mechanisms such as usage policies specifying authorization rules such as data usage rules. Exchange or sharing of data may be conducted according to predefined authorization mechanisms. The data provided by the interface of the data owner via the data providing service may be accessed by the interface of the data user according to the usage policies attached to the data provided by the interface of the data owner.

A participant acting as Data Owner may assume the role of the Data Provider. However, there may be cases in which the Data Provider may not coincide with the Data Owner, e.g., if the data is technically managed by a different entity than the Data Owner, such as in the case of a company using an external service to provide data, or if data management activities are handed over to a data trustee. In cases in which the Data Owner does not act as the Data Provider at the same time, the only activity of the Data Owner is to authorize a Data Provider to make its data available to be used by the interface of the Data Consumer.

The Data Provider interface makes data available for being shared or exchanged between a Data Owner and a Data Consumer. To facilitate a data request from a Data Consumer interface, the Data Provider interface may provide metadata, such as a description of datasets e.g. the syntax, serialization and/or semantics of data sources, a description of the provider or a description of consumers, to a Broker Service Provider (see below). A Broker Service Provider is not necessarily required for a Data Consumer interface and a Data Provider interface to establish a connection.

Exchanging or sharing data with a Data Consumer interface needs not necessarily be the only activity of the Data Provider. The Data Provider interface may log the details of the successful or unsuccessful completion of transactions at a Clearing House (see below). The data provider interface may facilitate billing or resolve a conflicts. Furthermore, the Data Provider interface may use Data Apps to check, enrich or transform the data.

The Data Consumer interface may receive data from a Data Provider interface. The Data Consumer interface may be the mirror entity of the Data Provider interface. The executable components of the Data Provider interface may be mirrored by the Data Consumer interface. Before the connection to a Data Provider interface can be established, the Data Consumer may search for existing datasets by making an inquiry at a Broker Service Provider. The Broker Service Provider may provide the required metadata for the Data Consumer interface to connect to a Data Provider. Such metadata may include the ID and/or authentication mechanism of the data provider interface. Alternatively or additionally, the Data Consumer interface may establish a connection to a Data Provider interface, e.g. with or without involving a Broker Service Provider. In cases in which the information to connect with the Data Provider interface is already known to the Data Consumer interface, the Data Consumer interface may request the data and optionally the corresponding metadata directly from the Data Provider interface. Like a Data Provider interface, the Data Consumer interface may log the details of a successful or unsuccessful data exchange transaction at a Clearing House, use Data Apps to check, enrich, transform, etc. the data received, or use a Service Provider interface to connect to International Data Spaces, if it does not deploy the technical infrastructure for participation itself.

Similar to the Data Owner being the legal entity that has the legal control over its data, the Data User is the legal entity that has the legal right to use the data of a Data Owner as specified by the usage policy. In most cases, the Data User is identical with the Data Consumer. However, there may be scenarios in which these roles are assumed by different participants.

The identity provider may act as an agent. It may include a Certification Authority, for example, managing digital certificates for the participants of the International Data Spaces, a Dynamic Attribute Provisioning Service (DAPS), for instance, managing the dynamic attributes of the participants, and a service named Dynamic Trust Monitoring (DTM), for instance, for continuous monitoring of the security and behavior of the network. It may be responsible for issuing technical identities to parties that have been approved to become Participants in the International Data Spaces. The Identity Provider may be instructed to issue identities based on approved roles (see above). The Identity Provider may also manage the PKI rollout. There are two separate PKI hierarchies: one for software signatures, for example, Software Signing Root CA, and one for the IDS Connectors, for example, Service Root CA. An entity is assigned with either an end certificate or a sub/root-CA certificate. The Identity Provider may act as an authentication and/or authorization service by incorporating the DAPS.

Each IDS Connector may run different services and communicate with other IDS Connectors. Using the PKI, an IDS Connector protects the persistent storage of its services and the communication with other IDS Connectors. In order to verify PKI signatures (e.g., for authentication, authorization or for Data Apps that were downloaded), the IDS Connector stores the trusted root certificates, for instance, Service Root CA and Software Signing Root CA, in a way their integrity is preserved.

App stores may provide data apps which can be run inside isolated containers of the IDS connectors. These are applications that can be deployed in IDS Connectors to execute tasks like transformation, aggregation or analytics on the data. App stores can be provided by IDS members and must be separately certified under IDS standards. An App Store has a service sub CA. The International Data Spaces Association signs a certificate signing request (CSR) in order to approve Apps or App Stores. The CSR identifies the App Store and makes it possible to sign the service CSRs from the IDS Connectors requesting apps. The IDS Connector creates a key pair for every App it downloads. The private key protects the App's persistent data. When downloading an App from the App Store, the IDS Connector creates a CSR using the public key. The App Store signs the CSR and issues a certificate. The IDS Connector uses this certificate to make sure that the App it is running and is valid.

Vocabulary providers may manage and offer “vocabularies”, preferably, including ontologies, reference data models, metadata elements, etc. which can be used to annotate and describe datasets. Vocabulary providers provide these, often domain-specific, vocabularies and their references to the IDS Information Model, which is the basis for the description of data sources.

The Broker may be used as an intermediary that stores and manages information about the data sources available in the International Data Spaces. As the role of the Broker is central but non-exclusive, multiple Brokers may be around at the same time e.g., for different application domains. An organization offering broker services in the International Data Spaces may assume other intermediary roles at the same time e.g., Clearing House or Identity Provider (see below). The activities of the Broker may include receiving and providing metadata. The Broker must provide an interface for Data Provider interfaces to send their metadata. The metadata may be stored in an internal repository for being queried by Data Consumers in a structured manner. While the core of the metadata model may be specified by the International Data Spaces, a Broker may extend the metadata model to manage additional metadata elements. After the Broker has provided the Data Consumer interface with the metadata about a certain Data Provider interface, its job is done, in particular, it is not involved in the subsequent data exchange process.

The Clearing House may be an intermediary that provides clearing and settlement services for financial and data sharing or exchange transactions. In the International Data Spaces, clearing activities are separated from broker services, since these activities are technically different from maintaining a metadata repository. As already stated above, it might still be possible that the two roles “Clearing House” and “Broker” are assumed by the same organization, as both roles require acting as a trusted intermediary between the Data Provider interface and the Data Consumer interface. The clearing house and the broker may include a distributed ledger to record metadata from data services. The Clearing House logs activities performed in the course of a data exchange. After a data exchange, or parts of it, are completed, both the Data Provider interface and the Data Consumer interface confirm the data transfer by logging the details of the transaction at the Clearing House. Based on this logging information, the transaction may be checked or billed. The logging information can also be used to resolve conflicts, e.g., to clarify whether a data package has been received by the Data Consumer or not. The Clearing House may provide reports on the performed (logged) transactions for billing, conflict resolution, etc.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the receiving of product data, the accessing of the semantic model, the generating of the product passport, the providing of the passport, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for generating a chemical product passport for a chemical product. One or more computing processors of the apparatus are configured to perform the following steps. Product data referring to information related to the product and/or one or more pre-products utilized in the production process of the product is received. Further, a semantic model comprising data related to at least a part of the product data is accessed. The chemical product passport is then generated based on the product data and the semantic model, wherein the chemical product passport comprises a decentralized identifier identifying the product, at least a part of the product data and a relation between at least a part of the product data provided by the chemical product passport and the semantic model

1. An apparatus for generating a chemical product passport for a chemical product, wherein the chemical product passport is indicative of a digital representation of data associated with the chemical product, wherein the apparatus comprises one or more computing processors configured to perform the followings steps:
receiving product data referring to information related to the product and/or one or more pre-products utilized in the production process of the product,
accessing a semantic model comprising data related to at least a part of the product data,
generating the chemical product passport based on the product data and the semantic model, wherein the chemical product passport comprises a decentralized identifier identifying the product, at least a part of the product data and a relation between at least a part of the product data provided by the chemical product passport and the semantic model, and
providing the generated product passport.

2. The apparatus according to claim 1, wherein the semantic model comprises a graph database in which the data related to at least a part of the product data is organized.

3. The apparatus according to claim 2, wherein the generating of the chemical product passport comprises selecting the product data provided by the chemical product passport based on a received request, wherein the received request is translated into a graph pattern request and the graph database of the semantic model is filtered for the graph pattern corresponding to the graph pattern request and the chemical data provided by the chemical product passport is selected based on the filtered graph pattern.

4. The apparatus according to claim 1, wherein the chemical product comprises a physical identifier and wherein the generating of this chemical product passport comprises assigning the physical identifier to the decentral identifier, wherein the product data refers to data collected in association with the physical identifier of the chemical product.

5. The apparatus according to claim 1, wherein the decentral identifier is further associated with a data schema and the chemical product passport is generated to comprise at least a part of the data schema and/or a data schema ID.

6. The apparatus according to claim 1, wherein the chemical product passport is associated with one or more authorization mechanisms that refer to the decentral identifier, the chemical product data provided by the chemical product passport and/or the access to the semantic model.

7. The apparatus according to claim 1, wherein the at least a part of the product data includes at least one of chemical product declaration data, chemical product safety data, material information and a certificate of analysis data associated with at least one physical entity of the chemical product.

8. A chemical product passport indicative of a digital representation of data associated with the chemical product, wherein the chemical product passport comprises a decentralized identifier identifying the product, at least a part of product data and a relation between at least a part of the product data provided by the chemical product passport and a semantic model, wherein the product data refers to information related to the product and/or one or more pre-products utilized in the production process of the product, and wherein the semantic model comprising data related to at least a part of the product data.

9. A semantic model utilizable for generating a chemical product passport for a chemical product according to claim 1, wherein the semantic model comprising data related to at least a part of product data referring to information related to the product and/or one or more pre-products utilized in the production process of the product, wherein the semantic model is configured such that a relation in the chemical product passport allows access to the semantic model.

10. A computer-implemented method for generating a chemical product passport, wherein the chemical product passport is indicative of a digital representation of data associated with a chemical product, wherein the method comprises:
receiving product data referring to information related to the product and/or one or more pre-products utilized in the production process of the product,
accessing a semantic model comprising data related to at least a part of the product data,
generating the chemical product passport based on the product data and the semantic model, wherein the chemical product passport comprises a decentralized identifier identifying the product, at least a part of the product data and a relation between at least a part of the product data provided by the chemical product passport and the semantic model, and
providing the generated product passport.

11. A computer program product for generating a chemical product passport comprising program code means for cause an apparatus according to 7 claim 1 to perform a method for generating a chemical product passport, wherein the chemical product passport is indicative of a digital representation of data associated with a chemical product, wherein the method comprises:
receiving product data referring to information related to the product and/or one or more pre-products utilized in the production process of the product,
accessing a semantic model comprising data related to at least a part of the product data,
generating the chemical product passport based on the product data and the semantic model, wherein the chemical product passport comprises a decentralized identifier identifying the product, at least a part of the product data and a relation between at least a part of the product data provided by the chemical product passport and the semantic model, and
providing the generated product passport.
